(19) United States
(12) Patent Application Publication
(10) Pub. No.: US 2010/0106119 A1
(43) Pub. Date: Apr. 29, 2010

(54) WASTE RECEPTACLE AND ATTACHMENT MEANS THEREFOR

(76) Inventor: LAURA DAYTON, PLACENTIA, CA (US)

Correspondence Address: FULWIDER PATTON LLP, HOWARD HUGHES CENTER, 6060 CENTER DRIVE, TENTH FLOOR, LOS ANGELES, CA, 90045, US

(21) Appl. No.: 12/258,207

(22) Filed: Oct. 24, 2008

(51) Int. Cl. A61M 1/00

(52) U.S. Cl. 604/322

(57) ABSTRACT

A waste system for use in conjunction with a patient's bed is described. The system comprises an elongate arm with first and second ends configured to permit the first end to be inserted between a mattress and a bed box of the bed. A receptacle for receiving human vomit is connected to the second end of the arm. A clamp is connected to the arm, configured to clamp onto a portion of the bed to prevent movement of the system in relation to the bed.

### BACKGROUND

This application relates to a receptacle for receiving human waste. Specifically, the application deals with a receptacle at a bedside of a patient, conveniently positioned to receive vomit of the patient.

The use of containers for bedridden patients to vomit into is known. Typically, a bedridden patient is provided with a container in the form of a bucket, typically having a plastic liner, to vomit into from the side of the bed. Such a bucket is usually set down next to the patient's bed, within his or her reach for use in an emergency. However, this procedure has many disadvantages. A bucket provided in this way may be moved during the course of attention to the patient by nursing staff or others, perhaps unintentionally, but without the knowledge of the patient. Thus, in an emergency, when the patient urgently needs to use the container, it may be found located just out of reach, with unfortunate results. Another disadvantage is that a bucket on the floor may be accidentally knocked over by personnel moving around the patient's bed while providing nursing services, again with unfortunate results.

Thus there is a need in the art for a convenient system and method for supplying a bedridden patient with a receptacle for vomiting into that will reliably remain in the same position in relation to the bed, within reach of the patient, and secure against accidentally being knocked over, yet which can be easily repositioned or removed entirely.

### SUMMARY OF THE INVENTION

According to a preferred embodiment of the invention, there is described a waste system configured to address various disadvantages in the prior art. The system includes an elongate arm with first and second ends configured to permit the first end to be inserted between a mattress and a bed box of the bed. A receptacle for receiving waste in the form of vomit is connected to the second end of the arm, so that the receptacle is positioned adjacent the bed, and held there by the arm extending under the mattress. In one aspect, a clamp may be connected to the arm, the clamp being configured to clamp onto a portion of the bed, either a bar which is a part of the bed, or the mattress of the bed. In another aspect of the invention, the receptacle is collapsible along an axis of the receptacle, and to this end the receptacle may comprise a plurality of concentric cylinders which collapse into the largest cylinder to reduce space during storage. In yet another aspect, the receptacle is connected to the arm by a hinge for permitting the collapsed receptacle to fold against the arm during storage. In yet a further aspect of the invention, the arm is telescopically extensible and/or retractable, which is advantageous for storage and also for setting the receptacle at exactly the desired distance from the bed.

In a preferred embodiment, the telescoping arm includes a first cylindrical portion and a second cylindrical portion slideable within the first cylindrical portion. In order to lock the two cylindrical portions in immovable relation to each other, a cap may be fixed to the first cylindrical portion configured to threadingly engage with a ring rotatable about the second cylindrical portion. The cap includes a conical forward portion and the ring includes an inverse conical portion mating with the conical portion. Additionally the conical portion may define a plurality of slots so that, when the ring is rotated about the cap, the engagement of the two sets of threads causes the ring to advance over the cap and reduce the diameter of the cap so as to clamp the cap onto the second cylindrical portion. This clamping effect holds the first and second cylindrical portions immovable in relation to each other.

A further aspect of the invention is that the clamp, described above, may be attached to the arm by a universal swivel. Preferably, the clamp is attached to the arm by a hoop positioned in a groove on the arm, and the hoop is sized to permit it to rotate about the arm. Desirably, the clamp is extensible in a radial direction with respect to the arm. These features provide flexibility to the clamp and allows a user to easily alter the position of the clamp in relation to the arm.

In a further facet, the invention includes a method of positioning a receptacle in fixed relation to a bed. The method comprises fixing the receptacle to an elongate arm, and inserting a portion of the arm between a mattress and a bed box of the bed, in order to allow the user to position the receptacle in a desired position adjacent to the bed while the receptacle is supported by the arm. The arm may be further attached to the bed with a clamp, thereby holding the receptacle in fixed relation to the bed. Desirably, inserting a portion of the arm between a mattress and a bed box includes telescopically adjusting the length of the arm to permit the best positioning of the receptacle in relation to the bed. The clamp may be attached to the bed either by clamping it onto a bar portion of the bed, or onto the mattress in the event no bar is provided.

These and other advantages of the invention will become more apparent from the following detailed description thereof and the accompanying exemplary drawings.

### BRIEF DESCRIPTION OF THE DRAWINGS

FIG. 1a is a perspective view of a waste system having features of the present invention, deployed in a typical configuration in relation to a bed.

FIG. 1b is a side view of the waste system of FIG. 1, shown in extended condition for use.

FIG. 2 is a partial section, partial elevation detail view of a feature of the waste system in FIG. 1.

FIG. 3 is a section view of a component of the waste system of FIG. 1.

FIG. 4 is a detail perspective view of an aspect of the waste system of FIG. 1.

FIG. 5 is a side view of a waste system of FIG. 1, shown in a folded condition for storage.

FIGS. 6a-d are schematic views of further embodiments of the invention.

### DETAILED DESCRIPTION OF THE PREFERRED EMBODIMENTS

With reference to the drawings, there is described a preferred embodiment of a system and method for providing a patient with a waste system having features of the present invention.

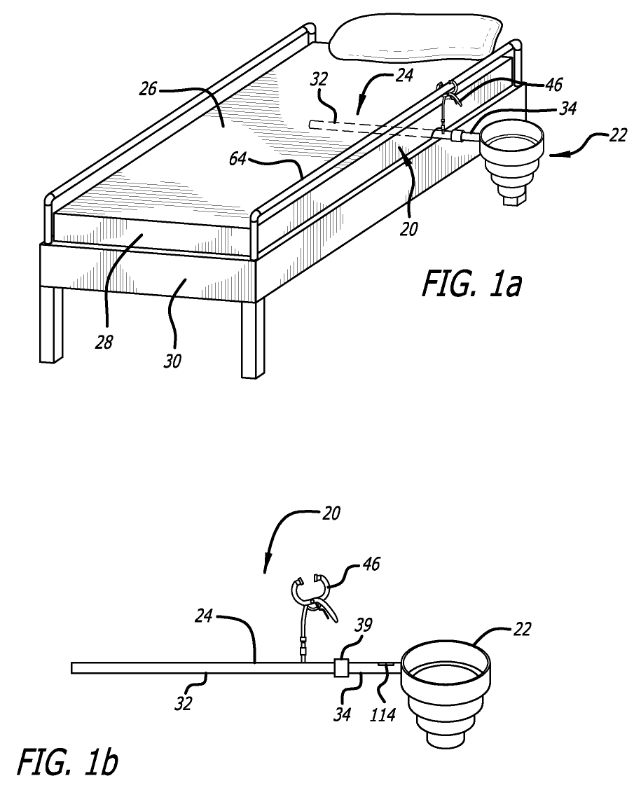

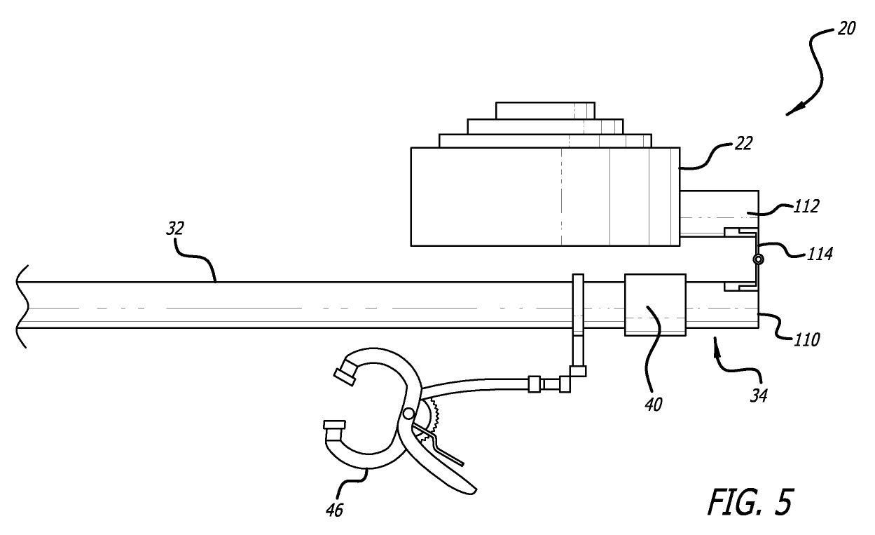

With reference to FIGS. 1 and 5 there is shown a waste system having features of the present invention generally identified by the numeral 20. In a preferred embodiment, the waste system includes a receptacle 22 attached to an arm 24 for receiving vomit. The system configuration allows the arm 24 to be inserted into a bed 26, preferably between the mattress 28 and the box 30 of the bed, so that the receptacle is positioned just to the side of the bed at a convenient height and position for a patient recumbent in the bed to vomit into in an emergency. This initial configuration gives an advantageous degree of fixture to the receptacle, so that it cannot be easily moved beyond the reach of the patient, who can rely upon waking up in an emergency to find the receptacle generally where it should be. At the same time, it is easily repositionable, and is easily removed entirely, while not being susceptible to being kicked over accidentally.

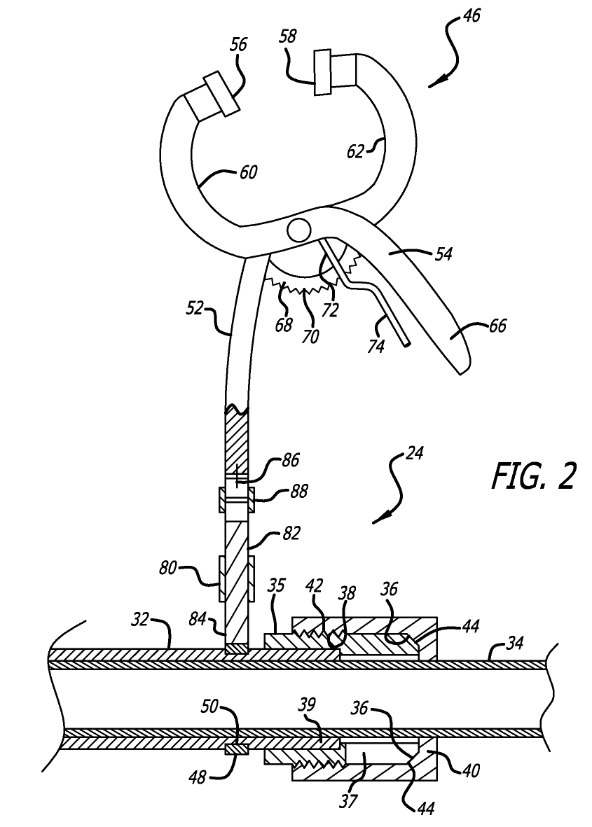

In a preferred embodiment, as exemplified in FIG. 2, and in order to provide greater versatility and security to the system, additional features may be provided. The arm 24 may be made to permit telescopic extension. Preferably, this feature is enabled by providing the arm 24 with a first hollow cylindrical portion 32, and at least a second cylindrical portion 34 slideable within the first cylindrical portion. To provide rotational and extensional stability between the two portions 32, 34, an open ended cylindrical cap 35 bearing external threads 38 is fixed to the proximal end 39 of the first portion. The cap preferably has an external conical shaped end point 36 extending beyond the first cylindrical portion and defining a plurality of slots 37 for allowing the end point 36 to radially expand and contract under an appropriate load. A ring 40 is provided to be slideable along the second cylindrical portion 34, the ring having internal threads 42 mating with the external threads 38 on the cap. The ring defines an internal cone 44 mating with the conical end point 36. When the ring 40 is threadingly engaged with and rotated about the cap 35, the ring advances along the cap so that the internal cone 44 bears down on the external end point 36 of the cap to reduce the diameter of the end point 36 and clamp the end point onto the second cylindrical portion 34 of the arm. Because the cap 35 is fixed to the first cylindrical portion 32, this clamping action has the effect of fixing the second cylindrical portion to the first cylindrical portion at any desired extension and degree of orientation. The user is thus advantageously able to set the length of the arm, and to set the distance of the receptacle 22 from the bed once the first cylindrical portion 32 of the arm 24 has been inserted between the mattress 28 and box 30 of the bed 26.

Again with reference to FIG. 2, there is shown an additional aspect of the invention. A clamp 46 is provided adjacent a proximal end 39 of the first cylindrical portion 32 of the arm. The clamp 46 allows a member of a nursing staff to removably fix the first cylindrical portion 32 of the arm in an immovable relation to the bed, while allowing her to adjust the amount by which the second cylindrical portion 34 extends away from the bed. The clamp 46 has the advantage of ensuring that the receptacle 22 remains in a stable position in relation to the bed, and is not susceptible to inadvertent movement by members of the nursing staff through bumping or by other causes. Immovability is advantageous in these circumstances once again because the patient is not surprised to find that the receptacle at a different position where he or she did not expect to find it; it has further benefit when the receptacle may be filled with vomit because inadvertent movement may cause the receptacle to empty its contents onto the floor. In a preferred embodiment, the clamp 46 is attached to the arm 24 near a forward, or proximal end 39 of the first cylindrical portion 32.

The clamp may include a number of advantageous features. Attachment of the clamp 46 to a forward end of the first cylindrical portion 32 is preferably achieved without penetrating the bore of the first cylindrical portion in order to allow the second cylindrical portion 34 to slide along the bore. This may be achieved by a hoop connection in which a hoop 48 is circumferentially tensioned onto the first cylindrical portion 32; preferably however, the hoop may be set in a slot 50 on the first cylindrical portion 32, allowing the hoop to rotate about, but not slide along, the first cylindrical portion 32. A first clasping member 52 may be connected to the hoop 48. A second clasping member 54 may be pivotably attached to the first clasping member 52. The first and second clasping members 52, 54 are shaped to clasp an object at their external tips 56, 58. Additionally each clasping member may have a curved portion 60, 62 suitable for clasping a bar 64 (FIG. 1) of between about 1 and 3 inches in diameter. A handle end 66 of the second clasping member opposite the tip 56 is suitable for use as a handle, allowing a user to manipulate the clasping members in relation to one another. In order to lock the first and second clasping members 52, 54 against movement relative to each other, a ratchet 68 bearing teeth 70 may be fixed to one clasping member 52, while a pivoting pawl 72 engageable with the ratchet teeth may be attached to the other clasping member 54. These features allow the clasping members 52, 54 to fixedly clamp onto any object that will fit between them, wherein the pawl engages with the teeth to prevent movement of the clasping members relative to each other. Preferably the pawl 72 is manually disengageable from the teeth 70 by a disengagement trigger 74, which allows a user to release the clamp from its fixed position as desired.

Thus, the clamp may be clamped onto a bar 64 (FIG. 1) extending along the edge of the bed 26 to hold the arm 24 in an immovable relation to the bed. Alternatively, where no bar is provided on the bed, the tips 56, 58 of the clamp may be made to clamp onto a portion of the mattress to produce the same effect. In a preferred embodiment, the first clasping member 52 is extensible, and may swivel and bend about its axis, to allow a user to position the clamp 46 at a wider range of positions in relation to the arm 24. The extension and swivel movement of the clamp may be provided by a turnbuckle 80 threadingly engaged with opposing threads 82, 84 on the first clasping member 52. Additionally, the first clasping member may flex about its axis as provided for by two independent pinned hinges 86, 88, or by a universal swivel joint.

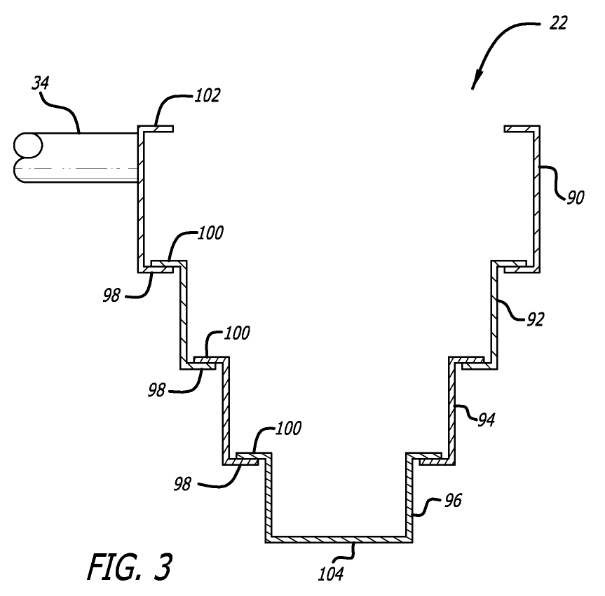

With reference to FIG. 3, the receptacle 22 at the end of the second cylindrical portion 34 of the arm 24 is described. In a preferred embodiment, the receptacle may be collapsible to facilitate storage of the entire system 20. Preferably, in order to achieve collapsibility, the receptacle 22 may be made up of a plurality of concentric cylinders 90, 92, 94, 96, each with an inwardly extending lower lip 98 and an outwardly extending upper lip 100. However, the upper lip 102 of the uppermost cylinder 90 may extend inwardly to a length sufficient to prevent the other cylindrical portions from falling through the largest diameter of the uppermost cylinder when the receptacle is inverted. Moreover, the lower lip 104 of the lowermost cylinder may be a continuous flat plate extending across the diameter of that cylinder to act as a support to any plastic bag inserted into the receptacle to provide a fluid-tight retention means for holding human waste in the form of vomit.

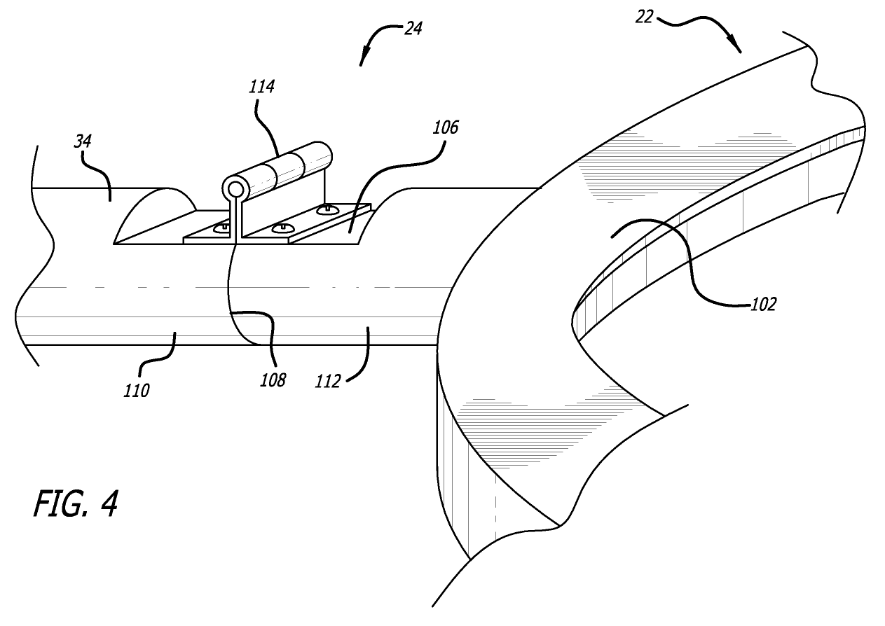

In a further aspect of the invention, exemplified in FIGS. 4 and 5, the receptacle 22 may be made to be foldable in relation to the arm 24. In this aspect, an upper surface of the second cylindrical portion 34 of the arm 24 is configured to have a flat surface 106, as seen in FIG. 4. The second cylindrical portion 34 is cut 108 through in the region of the flat surface 106 to produce two separated portions, a rear portion 110 and a forward portion 112. The rear and forward portions are then connected to each other by a hinge 114 that will allow the receptacle 22 to be folded against the arm 24 during storage. Thus, the hinged collapsible receptacle 22, and the telescoping arm 24 allow the entire system to be compactly reduced in axial length and radial extent for storage purposes, as may be seen in FIG. 5. When the receptacle is folded against the arm, the clamp 46 may be swiveled downwardly out of the way, or the hoop 48 may be rotated circumferentially to move the clamp 46 out of the way as shown in FIG. 5.

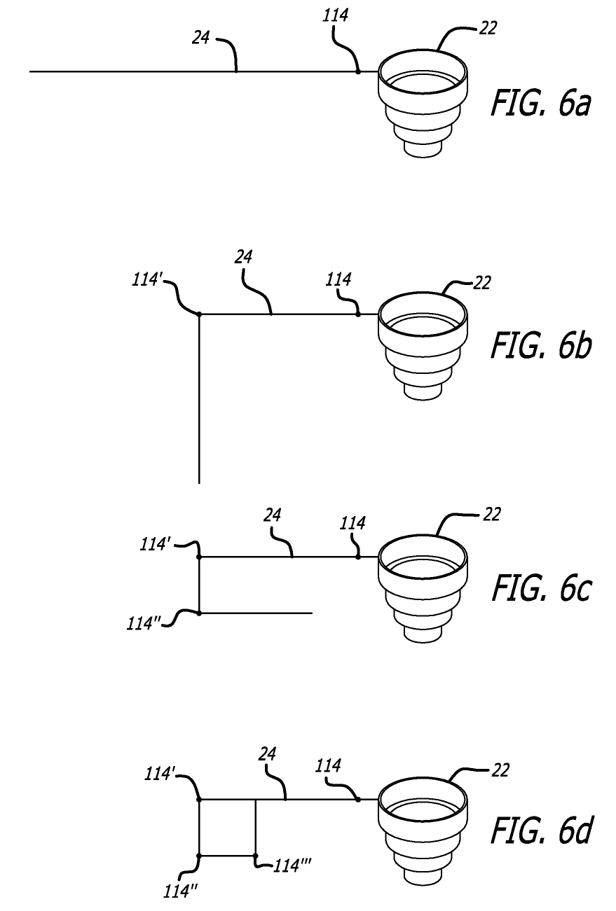

In a final aspect of the invention, the telescoping feature of the arm 24 may be omitted, and additional hinges 114′, 114″, 114′″ may be installed on the arm 24 of the invention. As schematically exemplified in FIG. 6a-d, which show a plurality of additional embodiments of the invention, these additional hinges allow the arm to be bent in a plurality of locations. This aspect facilitates the attachment of the device to structure in addition to beds, for example chairs, reclining chairs, wheel chairs, and the like. In such use, the arm 24 may be bent around such structure at points on the arm 24 at the location of the hinges 114′, 114″ and 114′″ that correspond with the profile of such structure, to provide a secure attachment to the structure. The same beneficial results follow, in which the receptacle 22 is located nearby a sick person who is confined to the chair, or other structure, so that when it is needed urgently, it is close at hand. The same structure may of course be used in conjunction with a bed, but the additional hinges provide for greater versatility and a wider range of uses.

Thus, it is seen that the waste system of the present invention provides novel and useful features for overcoming shortcomings in the prior art. The present invention may, of course, be carried out in other specific ways than those herein set forth without departing from the essential characteristics of the invention. The present embodiments are, therefore, to be considered in all respects as illustrative and not restrictive, and all changes coming within the meaning and equivalency range of the appended claims are intended to be embraced therein.

We claim:

1. A waste system for use in conjunction with a bed comprising:
an elongate arm with first and second ends configured to permit the first end to be inserted between a mattress and a bed box of the bed;
a receptacle for receiving vomit, the receptacle being connected to the second end of the arm;
a clamp connected to the arm, the clamp being configured to clamp onto a portion of the bed.

2. The waste system of claim 1, wherein the receptacle is collapsible along an axis of the receptacle.

3. The waste system of claim 2, wherein the receptacle comprises a plurality of concentric cylinders.

4. The waste system of claim 1, wherein the receptacle is connected to the arm by a hinge for permitting the receptacle to fold against the arm during storage.

5. The waste system of claim 1, wherein the arm is telescopically extensible.

6. The waste system of claim 5, wherein the arm includes a first cylindrical portion and a second cylindrical portion slideable within the first cylindrical portion, and further including a cap fixed to the first cylindrical portion configured to threadingly engage with a ring rotatable about the second cylindrical portion, wherein the cap includes a conical portion defining a plurality of slots, and the ring includes an inverse conical portion mating with the conical portion, whereby threaded rotation of the ring about the cap causes the ring to advance upon the cap so as to reduce the diameter of the cap and to clamp the cap onto the second cylindrical portion thereby holding the first and second cylindrical portions immovable in relation to each other.

7. The waste system of claim 1, wherein the clamp is attached to the arm by a swivel link.

8. The waste system of claim 1, wherein the clamp is attached to the arm by a hoop positioned in a groove on the arm, the hoop being sized in relation to the groove to freely rotate about the arm.

9. The waste system of claim 1, wherein the clamp is extensible in a radial direction with respect to the arm.

10. The waste system of claim 1, wherein the clamp includes two curved arms configured to clamp onto a cylindrical bar.

11. The waste system of claim 1, wherein the clamp includes two tips configured to opposingly clamp onto the mattress.

12. The waste system of claim 1, wherein the arm includes a plurality of hinges for allowing the arm to be bent around the profile of a structure.

13. A waste system for use in conjunction with a bed comprising:
an elongate arm with first and second ends configured to permit the first end to be inserted between a mattress and a bed box of the bed;
a receptacle for receiving vomit, the receptacle being connected to the second end of the arm;
wherein the receptacle is connected to the arm by a hinge for permitting the receptacle to fold against the arm during storage.

14. The waste system of claim 13, wherein the receptacle is collapsible along an axis of the receptacle.

15. The waste system of claim 14, wherein the receptacle comprises a plurality of concentric cylinders.

16. The waste system of claim 13, wherein the arm is telescopically extensible.

17. The waste system of claim 16, wherein the arm includes a first cylindrical portion and a second cylindrical portion slideable within the first cylindrical portion, and further including a cap fixed to the first cylindrical portion configured to threadingly engage with a ring rotatable about the second cylindrical portion, wherein the cap includes a conical portion defining a plurality of slots, and the ring includes an inverse conical portion mating with the conical portion, whereby threaded rotation of the ring about the cap causes the ring to advance upon the cap so as to reduce the diameter of the cap and to clamp the cap onto the second cylindrical portion thereby holding the first and second cylindrical portions immovable in relation to each other.

18. The waste system of claim 13, wherein the arm includes a plurality of hinges for allowing the arm to be bent around the profile of a structure.

19. A method of positioning a receptacle in fixed relation to a bed comprising:
fixing the receptacle to an elongate arm;
inserting a portion of the arm between a mattress and a bed box of the bed, thereby allowing the receptacle to be positioned adjacent to the bed;
attaching the arm to the bed with a clamp, thereby holding the receptacle in fixed relation to the bed;

20. The method of claim 18, wherein inserting a portion of the arm between a mattress and a bed box includes telescopically adjusting the length of the arm.

21. The method of claim 18, wherein attaching the arm to the bed with a clamp includes attaching the clamp onto a bar portion of the bed.

22. The method of claim 18, wherein attaching the arm to the bed with a clamp includes attaching the clamp to the mattress.
